(19) United States
(12) Patent Application Publication
(10) Pub. No.: US 2006/0039952 A1
(43) Pub. Date: Feb. 23, 2006

(54) Ophthalmic drug delivery device

(76) Inventor: Yoseph Yaacobi, Fort Worth, TX (US)

Correspondence Address: ALCON RESEARCH, LTD., R&D COUNSEL, Q-148, 6201 SOUTH FREEWAY, FORT WORTH, TX, 76134-2099, US

(21) Appl. No.: 11/248,727

(22) Filed: Oct. 12, 2005

Related U.S. Application Data
(63) Continuation of application No. PCT/US04/20087, filed on Jun. 23, 2004.
(60) Provisional application No. 60/485,995, filed on Jul. 10, 2003.

(51) Int. Cl. A61F 2/00

(52) U.S. Cl. 424/427

(57) ABSTRACT

Ophthalmic drug delivery devices useful for delivery of pharmaceutically active agents to the posterior segment of the eye are disclosed. The devices may include extensions, immobilizing structures, and/or geometries to help properly locate, and prevent migration of, the devices.

This application is a continuation of PCT/US2004/020087 filed Jun. 23, 2004 entitled “Ophthalmic Drug Delivery Device,” which claims priority from U.S. Provisional Application No. 60/485,995 filed Jul. 10, 2003.

### FIELD OF THE INVENTION

The present invention generally pertains to biocompatible implants for localized delivery of pharmaceutically active agents to body tissue. More particularly, but not by way of limitation, the present invention pertains to biocompatible implants for localized delivery of pharmaceutically active agents to the posterior segment of the eye.

### DESCRIPTION OF THE RELATED ART

Several diseases and conditions of the posterior segment of the eye threaten vision. Age related macular degeneration (ARMD), choroidal neovascularization (CNV), retinopathies (e.g., diabetic retinopathy, vitreoretinopathy), retinitis (e.g., cytomegalovirus (CMV) retinitis), uveitis, macular edema, glaucoma, and neuropathies are several examples.

Age related macular degeneration (ARMD) is the leading cause of blindness in the elderly. ARMD attacks the center of vision and blurs it, making reading, driving, and other detailed tasks difficult or impossible. About 200,000 new cases of ARMD occur each year in the United States alone. Current estimates reveal that approximately forty percent of the population over age 75, and approximately twenty percent of the population over age 60, suffer from some degree of macular degeneration. “Wet” ARMD is the type of ARMD that most often causes blindness. In wet ARMD, newly formed choroidal blood vessels (choroidal neovascularization (CNV)) leak fluid and cause progressive damage to the retina.

In the particular case of CNV in ARMD, three main methods of treatment are currently being developed, (a) photocoagulation, (b) the use of angiogenesis inhibitors, and (c) photodynamic therapy. Photocoagulation is the most common treatment modality for CNV. However, photocoagulation can be harmful to the retina and is impractical when the CNV is near the fovea. Furthermore, over time, photocoagulation often results in recurrent CNV. Oral or parenteral (non-ocular) administration of anti-angiogenic compounds is also being tested as a systemic treatment for ARMD. However, due to drug-specific metabolic restrictions, systemic administration usually provides sub-therapeutic drug levels to the eye. Therefore, to achieve effective intraocular drug concentrations, either an unacceptably high dose or repetitive conventional doses are required. Periocular injections of these compounds often result in the drug being quickly washed out and depleted from the eye, via periocular vasculature and soft tissue, into the general circulation. Repetitive intraocular injections may result in severe, often blinding, complications such as retinal detachment and endophthalmitis. Photodynamic therapy is a new technology for which the long-term efficacy is still largely unknown.

In order to prevent complications related to the above-described treatments and to provide better ocular treatment, researchers have suggested various implants aimed at localizing delivery of anti-angiogenic compounds to the eye. U.S. Pat. No. 5,824,072 to Wong discloses a non-biodegradable polymeric implant with a pharmaceutically active agent disposed therein. The pharmaceutically active agent diffuses through the polymer body of the implant into the target tissue. The pharmaceutically active agent may include drugs for the treatment of macular degeneration and diabetic retinopathy. The implant is placed substantially within the tear fluid upon the outer surface of the eye over an avascular region, and may be anchored in the conjunctiva or sclera; episclerally or intrasclerally over an avascular region; substantially within the suprachoroidial space over an avascular region such as the pars plana or a surgically induced avascular region; or in direct communication with the vitreous.

U.S. Pat. No. 5,476,511 to Gwon et al. discloses a polymer implant for placement under the conjunctiva of the eye. The implant may be used to deliver neovascular inhibitors for the treatment of ARMD and drugs for the treatment of retinopathies, and retinitis. The pharmaceutically active agent diffuses through the polymer body of the implant.

U.S. Pat. No. 5,773,019 to Ashton et al. discloses a non-bioerodable polymer implant for delivery of certain drugs including angiostatic steroids and drugs such as cyclosporine for the treatment of uveitis. Once again, the pharmaceutically active agent diffuses through the polymer body of the implant.

All of the above-described implants require careful design and manufacture to permit controlled diffusion of the pharmaceutically active agent through a polymer body (i.e., matrix devices) or polymer membrane (i.e., reservoir devices) to the desired site of therapy. Drug release from these devices depends on the porosity and diffusion characteristics of the matrix or membrane, respectively. These parameters must be tailored for each drug moiety to be used with these devices. Consequently, these requirements generally increase the complexity and cost of such implants.

U.S. Pat. No. 5,824,073 to Peyman discloses an indentor for positioning in the eye. The indentor has a raised portion that is used to indent or apply pressure to the sclera over the macular area of the eye. This patent discloses that such pressure decreases choroidal congestion and blood flow through the subretinal neovascular membrane, which, in turn, decreases bleeding and subretinal fluid accumulation.

Therefore, a need exists in the biocompatible implant field for a surgically implantable ophthalmic drug delivery device capable of safe, effective, rate-controlled, localized delivery of a wide variety of pharmaceutically active agents. The surgical procedure for implanting such a device should be safe, simple, quick, and capable of being performed in an outpatient setting. Ideally, such a device should be easy and economical to manufacture. Furthermore, because of its versatility and capability to deliver a wide variety of pharmaceutically active agents, such an implant should be capable of use in ophthalmic clinical studies to deliver various agents that create a specific physical condition in a patient. Such an ophthalmic drug delivery device is especially needed for localized delivery of pharmaceutically active agents to the posterior segment of the eye to combat ARMD, CNV, retinopathies, retinitis, uveitis, macular edema, glaucoma, and neuropathies.

### SUMMARY OF THE INVENTION

One aspect of the present invention is a drug delivery device for an eye. The eye has a sclera, a macula, and an extraocular muscle. The device includes a pharmaceutically active agent and a body having an extension for accomodating the extraocular muscle. When the device is disposed on an outer surface of the sclera so that the extension accomodates the extraocular muscle, the pharmaceutically active agent is disposed proximate the macula.

Another aspect of the present invention is a drug delivery device for an eye having a pharmaceutically active agent and a body having an extension for accomodating an extraocular muscle. When the device is disposed on an outer surface of the sclera so that the extension accomodates the extraocular muscle, the extension helps to immobilize and to prevent migration of the device.

A further aspect of the present invention is a drug delivery device for an eye having a pharmaceutically active agent and a body having an extension for accomodating an extraocular muscle. The extension is capable of extending from the body in a first position so as to accommodate the extraocular muscle. The extension is also capable of folding above or beneath the body in a second position so as to facilitate implantation of the device.

A further aspect of the present invention is a drug delivery device for an eye having a pharmaceutically active agent and a body having a scleral surface for contacting the sclera. An immobilizing structure is disposed on the scleral surface.

A further aspect of the present invention is a drug delivery device for an eye. The device has a body including a scleral surface, a well having an opening to the scleral surface, and a geometry that facilitates an implantation of the device on an outer surface of the sclera, between the superior rectus muscle and the lateral rectus muscle, beneath the lateral rectus muscle, and with the well disposed proximate the macula. The device also includes an inner core disposed in the well and comprising a pharmaceutically active agent.

A further aspect of the present invention is a method of delivering a pharmaceutically active agent to an eye. A drug delivery device is provided that includes a pharmaceutically active agent and a body having a geometry that facilitates an implantation of the device on an outer surface of the sclera, between the superior rectus muscle and the lateral rectus muscle, beneath the lateral rectus muscle, and with the pharmaceutically active agent disposed proximate the macula. The device is then disposed on an outer surface of the sclera, between the superior rectus muscle and the lateral rectus muscle, beneath the lateral rectus muscle, and with the pharmaceutically active agent disposed proximate the macula.

### BRIEF DESCRIPTION OF THE DRAWINGS

For a more complete understanding of the present invention, and for further objects and advantages thereof, reference is made to the following description taken in conjunction with the accompanying drawings in which:

FIG. 1 is a side sectional view schematically illustrating the human eye and an ophthalmic drug delivery device implanted in the posterior segment of the eye according to the present invention;

FIG. 2 is detailed cross-sectional view of the eye of FIG. 1 along line 2-2;

FIG. 3 is a lateral schematic view of the topographic anatomy of the extraocular muscles of a human eye;

FIG. 4 is a postero-lateral view of the topographical anatomy of the extraocular muscles of a human eye with a portion of the lateral rectus muscle not shown;

FIG. 5 is a perspective view of an orbital surface of an ophthalmic drug delivery device according to a first preferred embodiment of the present invention;

FIG. 6 is a perspective of a scleral surface of the ophthalmic drug delivery device of FIG. 5;

FIG. 7 is perspective view of a first side of the ophthalmic drug delivery device of FIG. 5;

FIG. 8 is a perspective view of a second side of the ophthalmic drug delivery device of FIG. 5;

FIG. 9 is a perspective view of a distal end of the ophthalmic drug delivery device of FIG. 5;

FIG. 10 is a perspective view of a proximal end of the ophthalmic drug delivery device of FIG. 5;

FIG. 11 is a schematic view of the ophthalmic drug delivery device of FIG. 5 in situ in a human eye;

FIGS. 12A-E schematically illustrate the implantation of the ophthalmic drug delivery device of FIG. 5 in the human eye according to a preferred method of the present invention;

FIG. 13 is a schematic view of an ophthalmic drug delivery device according to a second preferred embodiment of the present invention in situ in a human eye;

FIG. 14 is a schematic view of an ophthalmic drug delivery device according to a third preferred embodiment of the present invention in situ in a human eye; and

FIG. 15 is a schematic view of an ophthalmic drug delivery device according to a fourth preferred embodiment of the present invention in situ in a human eye;

### DETAILED DESCRIPTION OF THE PREFERRED EMBODIMENTS

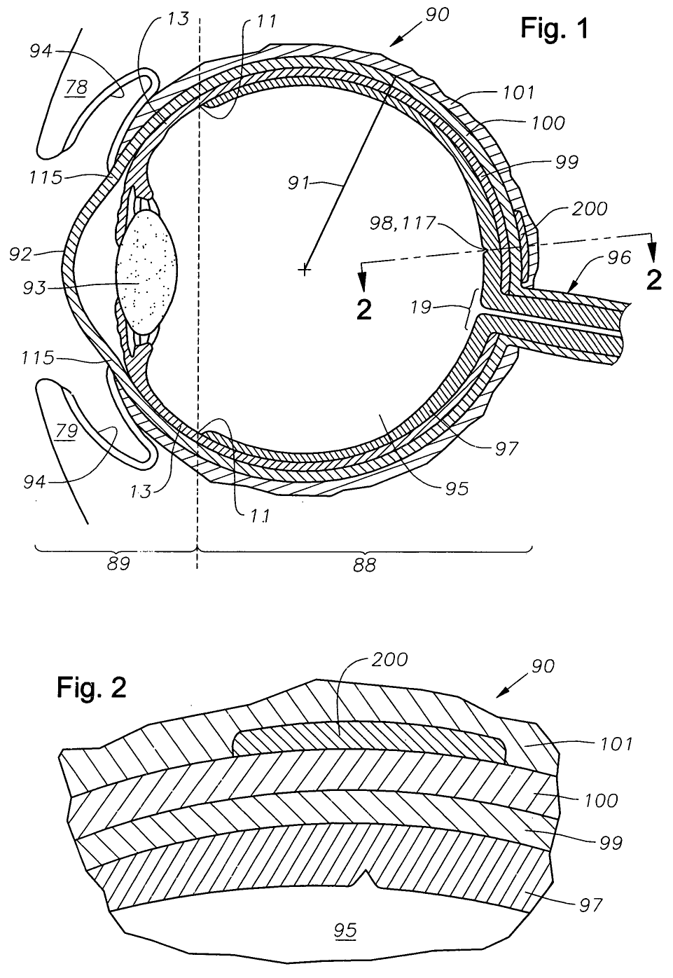

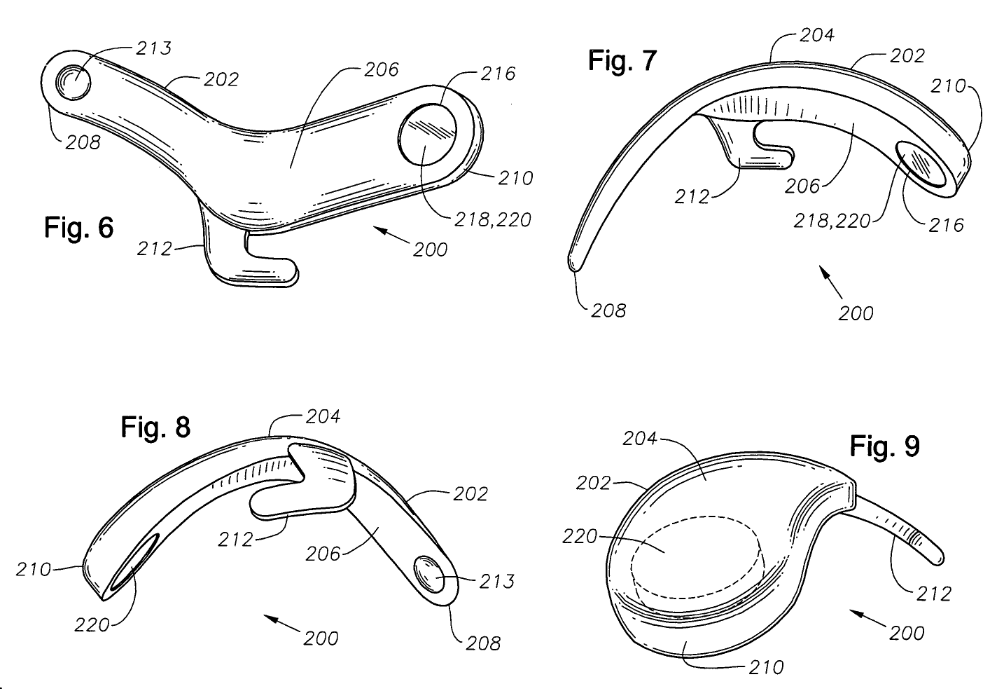

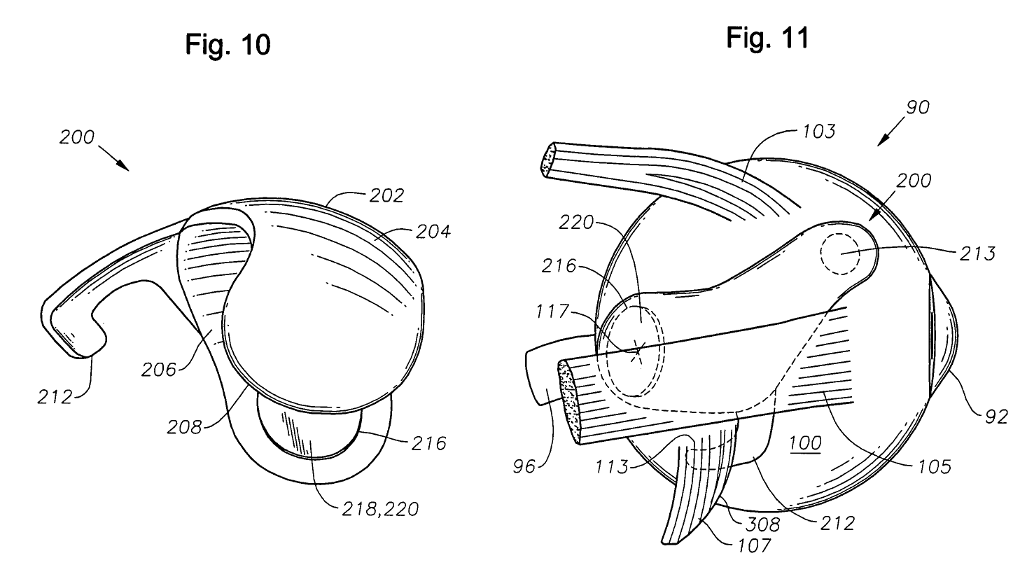

The preferred embodiments of the present invention and their advantages are best understood by referring to FIGS. 1-15 of the drawings, like numerals being used for like and corresponding parts of the various drawings.

FIGS. 1-4 illustrate various portions of the human eye important to a complete understanding of the present invention. Referring first to FIG. 1, a human eye 90 is schematically illustrated. Eye 90 has a cornea 92, a lens 93, vitreous 95, a sclera 100, a choroid 99, a retina 97, and an optic nerve 96. Eye 90 is generally divided into an anterior segment 89 and a posterior segment 88. Anterior segment 89 of eye 90 generally includes the portions of eye 90 anterior of ora serata 11. Posterior segment 88 of eye 90 generally includes the portions of eye 90 posterior of ora serata 11. Retina 97 is physically attached to choroid 99 in a circumferential manner proximate pars plana 13, posteriorly to optic disk 19. Retina 97 has a macula 98 located slightly lateral to optic disk 19. As is well known in the ophthalmic art, macula 98 is comprised primarily of retinal cones and is the region of maximum visual acuity in retina 97. At the center of macula 98 is a fovea 117. A Tenon's capsule or Tenon's membrane 101 is disposed on sclera 100. A conjunctiva 94 covers a short area of the globe of eye 90 posterior to limbus 115 (the bulbar conjunctiva) and folds up (the upper cul-de-sac) or down (the lower cul-de-sac) to cover the inner areas of upper eyelid 78 and lower eyelid 79, respectively. The bulbar conjunctiva 94 is disposed on top of Tenon's capsule 101.

As is shown in FIGS. 1 and 2, and as is described in greater detail hereinbelow, an ophthalmic drug delivery device 200 is preferably disposed directly on the outer surface of sclera 100, below Tenon's capsule 101 for treatment of most posterior segment diseases or conditions. In addition, for treatment of ARMD and CNV in humans, device 200 is preferably disposed directly on the outer surface of sclera 100, below Tenon's capsule 101, with an inner core of device 200 proximate macula 98. While device 200 is especially designed for use in humans, it may also be used in animals.

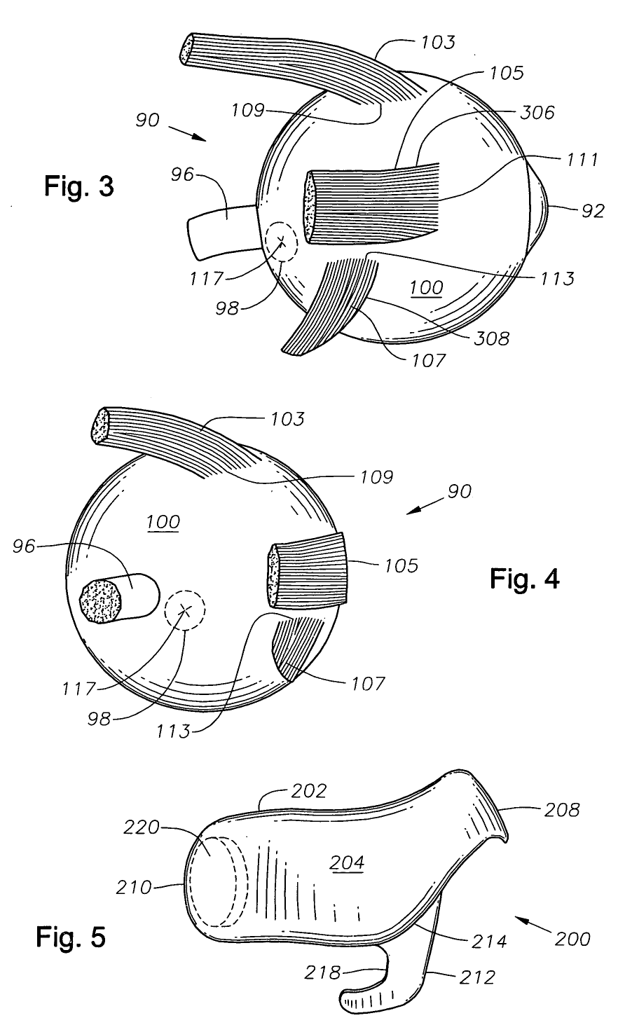

FIG. 3 schematically illustrates a topographical, lateral view of a right human eye 90 with its cornea 92, optic nerve 96, macula 98, sclera 100, superior rectus muscle 103, lateral rectus muscle 105, inferior oblique muscle 107, and fovea 117. Superior rectus muscle 103 has an insertion 109 into sclera 100. Lateral rectus muscle 105 has an insertion 111 into sclera 100. Inferior oblique muscle 107 has an insertion 113 into sclera 100. FIG. 4 schematically illustrates a topographical, postero-lateral view of right human eye 90 with a portion of lateral rectus muscle 105 truncated to allow visibility to the portion of sclera 100 hidden by the muscle.

FIGS. 5-10 schematically illustrate an ophthalmic drug delivery device 200 for the right human eye according to a first preferred embodiment of the present invention. An ophthalmic drug delivery device that is a mirror image of device 200 may be utilized for the left human eye. Device 200 may be used in any case where localized delivery of a pharmaceutically active agent to the eye is required. Device 200 is particularly useful for localized delivery of pharmaceutically active agents to the posterior segment of the eye. A preferred use for device 200 is the delivery of pharmaceutically active agents to the retina proximate the macula for treating ARMD, choroidial neovascularization (CNV), retinopathies, retinitis, uveitis, macular edema, glaucoma, and neuropathies.

Device 200 generally includes a body 202 having a convex, dome-shaped, orbital surface 204 and a concave, dome-shaped, scleral surface 206. Scleral surface 206 is designed with a radius of curvature that facilitates direct contact with sclera 100. Most preferably, scleral surface 206 is designed with a radius of curvature equal to the radius of curvature 91 of an average human eye 90. (See FIG. 1) Orbital surface 204 is preferably designed with a radius of curvature that facilitates implantation under Tenon's capsule 101. Device 200 has a proximal end 208, a distal end 210, an extension 212 extending from body 202, and an immobilizing structure 213 on scleral surface 206. Extension 212 is preferably integrally formed with body 202. Extension 212 is preferably foldable along line 214 so that the entire extension 212 may be folded underneath body 202 of device 200. Alternatively, device 200 may be designed so that the entire extension 212 may be folded above body 202. As is described in more detail hereinbelow, extension 212 is designed to accommodate insertion 113 of inferior oblique muscle 107 during implantation. Immobilizing structure 213 is preferably a suction cup and is preferably integrally formed on scleral surface 206. Alternatively, immobilizing structure 213 may be a bioadhesive coating or a region of one or more sharp prongs, if desired. As is described in more detail hereinbelow, immobilizing structure 213 mates with sclera 100 to help prevent migration of device 200 after implantation. Still further in the alternative, device 200 may be sutured to sclera 100, preferably near its proximal end 208, to immobilize the device and help prevent migration after implantation.

Device 200 also has a well or cavity 216 having an opening 218 to scleral surface 206. An inner core 220 is preferably disposed in well 216. As shown in FIGS. 5-10, inner core 220 is preferably a tablet comprising one or more pharmaceutically active agents. Alternatively, inner core 220 may comprise a conventional hydrogel, gel, paste, or other semi-solid dosage form having one or more pharmaceutically active agents disposed therein. Although not shown in FIGS. 5-10, inner core 220 may alternatively comprise a suspension, solution, powder, or combination thereof containing one or more pharmaceutically active agents. In this embodiment, scleral surface 206 is formed without opening 218, and the suspension, solution, powder, or combination thereof diffuses through a relatively thin extension of scleral surface 206 or other membrane below inner core 220. Still further in the alternative, device 200 may be formed without well 216 or inner core 220, and the pharmaceutically active agent(s) in the form of a suspension, solution, powder, or combination thereof may be dispersed throughout body 202 of device 200. In this embodiment, the pharmaceutically active agent diffuses through body 202 into the target tissue. The structure of well 216 and inner core 220 is more fully described in U.S. Pat. No. 6,413,540, which is hereby incorporated herein in its entirety by reference.

The geometry and dimensions of device 200 maximize communication between the pharmaceutically active agent of inner core 220 and the tissue underlying scleral surface 206. Scleral surface 206 preferably physically contacts the outer surface of sclera 100. Alternatively, scleral surface 206 may be disposed proximate the outer surface of sclera 100. By way of example, device 200 may be disposed in the periocular tissues just above the outer surface of sclera 100 or intra-lamellarly within sclera 100.

Body 202 preferably comprises a biocompatible, non-bioerodable material. Body 202 more preferably comprises a biocompatible, non-bioerodable polymeric composition. Said polymeric composition most preferably comprises silicone. Of course, said polymeric composition may also comprise other conventional materials that affect its physical properties, including, but not limited to, porosity, tortuosity, permeability, rigidity, hardness, and smoothness. Body 202 is preferably impermeable to the pharmaceutically active agent of inner core 220. Polymeric compositions, and conventional materials that affect their physical properties, suitable for body 202 are more fully disclosed in U.S. Pat. No. 6,416,777, which is hereby incorporated herein in its entirety by reference.

Inner core 220 may comprise any ophthalmically acceptable pharmaceutically active agent suitable for localized delivery. Examples of pharmaceutically active agents suitable for inner core 220 are disclosed in U.S. Pat. No. 6,416,777. One preferred pharmaceutically active agent is angiostatic steroids for the prevention or treatment of diseases or conditions of the posterior segment of the eye, including, without limitation, ARMD, CNV, retinopathies, retinitis, uveitis, macular edema, and glaucoma. Such angiostatic steroids are more fully disclosed in U.S. Pat. Nos. 5,679,666 and 5,770,592, which are hereby incorporated herein in their entirety by reference. Preferred ones of such angiostatic steroids include 4,9(11)-Pregnadien-17α,21-diol-3,20-dione and 4,9(11)-Pregnadien-17α,21-diol-3,20-dione-21-acetate. In addition, inner core 220 may include a combination of a glucocorticoid and an angiostatic steroid as pharmaceutically active agents. For this combination, preferred glucocorticoids include dexamethasone, fluoromethalone, medrysone, betamethasone, triamcinolone, triamcinolone acetonide, prednisone, prednisolone, hydrocortisone, rimexolone, and pharmaceuitcally acceptable salts thereof, and preferred angiostatic steroids include 4,9(11)-Pregnadien-17α,21-diol-3,20-dione and 4,9(11)-Pregnadien-17α,21-diol-3,20-dione-21-acetate. Inner core 220 may also comprise conventional non-active excipients to enhance the stability, solubility, penetrability, or other properties of the active agent or the drug core. If inner core 220 is a tablet, it may further comprise conventional excipients necessary for tableting, such as fillers and lubricants. Such tablets may be produced using conventional tableting methods. The pharmaceutically active agent is preferably distributed evenly throughout the tablet. In addition to conventional tablets, inner core 220 may comprise a special tablet that bioerodes at a controlled rate, releasing the pharmaceutically active agent. By way of example, such bioerosion may occur through hydrolosis or enzymatic cleavage. If inner core 220 is a hydrogel or other gel, such gels may bioerode at a controlled rate, releasing the pharmaceutically active agent. Alternatively, such gels may be non-bioerodable but allow diffusion of the pharmaceutically active agent.

Device 200 may be made by conventional polymer processing methods, including, but not limited to, injection molding, extrusion molding, transfer molding, and compression molding. Preferably, device 200 is formed using conventional injection molding techniques. Inner core 220 is preferably disposed in well 216 after the formation of body 202 of device 200.

As shown in FIG. 11, device 200 is preferably surgically placed directly on the outer surface of sclera 100 below Tenon's capsule 101 with well 216 and inner core 220 directly over the area of sclera 100 above macula 98. Most preferably, inner core 220 is directly over the area of sclera 100 above fovea 117, which is the center of macula 98. Extension 212 is disposed on the outer surface of sclera 100 and beneath the inferior oblique muscle 107 proximate to, or contacting, insertion 109 of the inferior oblique muscle 107. Due to the geometry of device 200, anchoring extension 212 to inferior oblique muscle 107 in this manner automatically locates inner core 220 over macula 98 and fovea 117. Anchoring extension 212 to inferior oblique muscle 107 in this manner also helps to immobilize and prevent migration of device 200 after implantation. Suction cup 213 is also gently applied to sclera 100 and further helps to immobilize and prevent migration of device 200 after implantation.

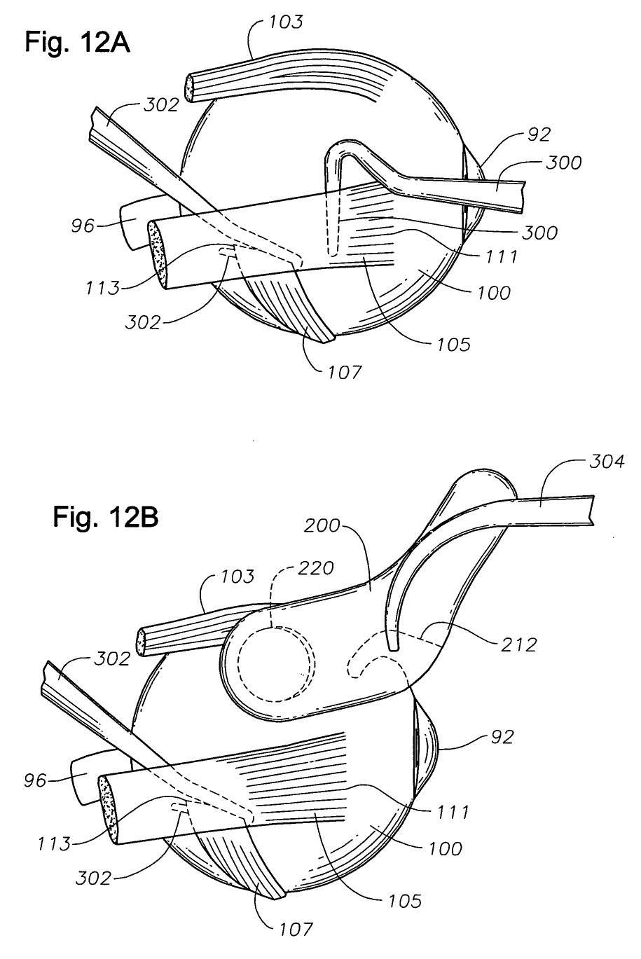

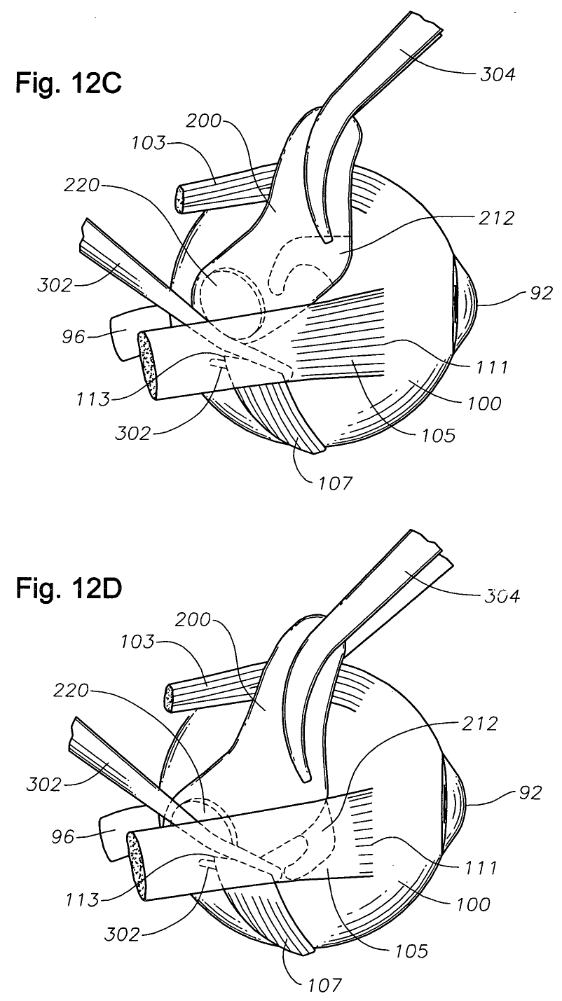

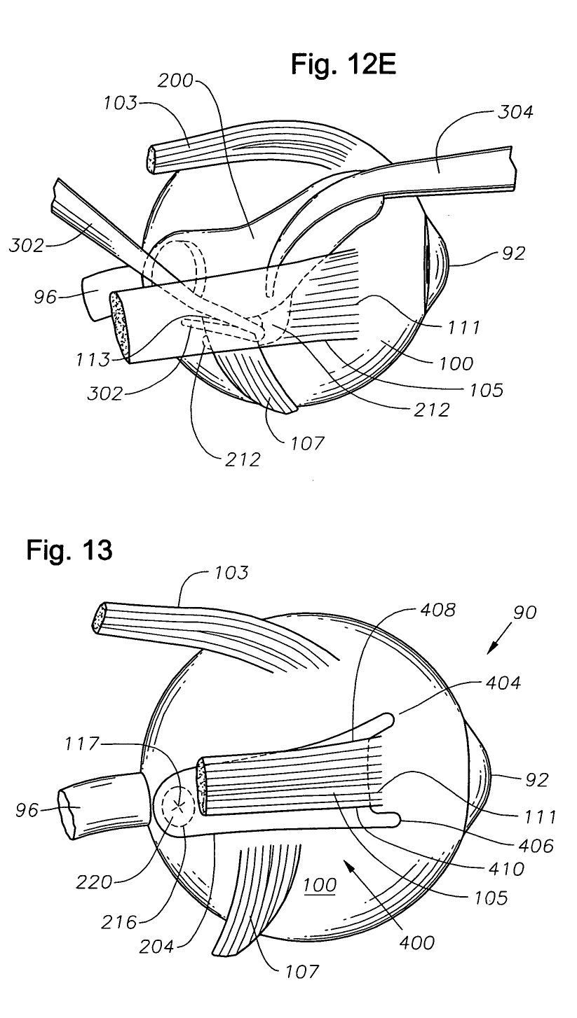

Referring generally to FIGS. 12A-E, the following technique, which is capable of being performed in an outpatient setting, is preferably utilized to implant device 200 into the position shown in FIG. 11. The surgeon first performs a circumferential peritomy in one of the quadrants of eye 90. Preferably, the surgeon performs the peritomy in the supero-temporal quadrant, about 3 mm posterior to limbus 115 of eye 90. Once this incision is made, the surgeon performs a blunt dissection to separate Tenon's capsule 101 from sclera 100. Using scissors and blunt dissection, an antero-posterior tunnel is formed along the outer surface of sclera 100 following the superior border 306 (FIG. 3) of lateral rectus muscle 105. The lateral rectus muscle 105, and then the inferior oblique muscle 107, are engaged with Jamison muscle hooks 300 and 302, respectively, and manipulated as shown in FIG. 12A. The hooks 300 and 302 are also used to gently break the connnective tissues between muscles 105 and 107 and sclera 100, further defining the tunnel for device 200. After removing the hook 300, the surgeon grasps device 200 with Nuggett forceps 304, as shown in FIG. 12B. Extension 212 is preferably folded beneath body 202 and held in this position with forceps 304. Using a device 200 with an extension 212 folded along line 214 minimizes the size of the peritomy and the tunnel required for device 200. Alternatively, a device 200 with an unfolded extension 212 may be utilized, if desired. With scleral surface 206 facing sclera 100 and distal end 210 away from the surgeon, the surgeon introduces device 200 into the tunnel using a generally circular motion, as shown by FIGS. 12C-E. When the surgeon visualizes that folded extension 212 has traveled past insertion 111 of lateral rectus muscle 105, he or she loosens forceps 304. This loosening of forceps 304 releases extension 212 and allows it to unfold, as shown in FIG. 12D. The surgeon then continues moving device 200 in a generally circular manner within the tunnel until extension 212 hooks under inferior oblique muscle 107, as shown in FIG. 12E. Preferably, anterior edge 218 (FIG. 5) of extension 212 contacts anterior border 308 (FIG. 3) and/or insertion 113 of inferior oblique muscle 107. If a non-foldable extension 212 is utilized, extension 212 is simply moved under anterior border 308 of inferior oblique muscle 107 in a similar manner. Although not shown in FIG. 12E, extension 212 may also be placed between hook 302 and insertion 113 of inferior oblique muscle 107. The surgeon then removes hook 302. Device 200 is then disposed in the position shown in FIG. 11. The surgeon uses forceps 304 to gently press orbital surface 204 near proximal end 208, securing suction cup 213 to sclera 100. Alternatively, the surgeon may use forceps 304 to gently press orbital surface 204 near proxmial end 208 to secure bioadhesive coating 213 or region of one or more sharp prongs 213 to sclera 100. Still further in the alternative, the surgeon may suture proximal end 208 of device 200 to sclera 100. The surgeon then closes the peritomy by suturing Tenon's capsule 101 and conjunctiva 94 to sclera 100. After closing, the surgeon places a strip of antibiotic ointment on the surgical wound.

The geometry of body 202 of device 200, including the concave nature of scleral surface 206; the shape and locations of extension 212, well 216, opening 218, and inner core 220; the presence of suction cup, bioadhesive coating, or region of sharp prong(s) 213, and the foldable nature of extension 212 all facilitate the delivery of a pharmaceutically effective amount of the pharmaceutically active agent from inner core 220 through sclera 100, choroid 99, and into retina 97, and more particularly into macula 98 and fovea 117. The absence of a polymer layer or membrane between inner core 220 and sclera 100 also greatly enhances and simplifies the delivery of an active agent to retina 97.

It is believed that device 200 can be used to deliver a pharmaceutically effective amount of a pharmaceutically active agent to retina 97 for many years, depending on the particular physicochemical properties of the pharmaceutically active agent employed. Important physicochemical properties include hydrophobicity, solubility, dissolution rate, diffusion coefficient, partitioning coefficient, and tissue affinity. After inner core 220 no longer contains active agent, the surgeon may easily remove device 200. In addition, the surgeon may use the “pre-formed” tunnel for the replacement of an old device 200 with a new device 200.

FIGS. 13-15 schematically illustrate ophthalmic drug delivery devices 400, 500, and 600 according to second, third, and fourth preferred embodiments of the present invention, respectively, in situ in the human eye. Each of devices 400, 500, and 600 are substantially similar in structure, operation, and use to device 200, except that the body of each of the devices has a different geometry when viewed from its orbital surface, and several of the devices have different extension(s) designed to accommodate a different extraocular muscle than extension 212 of device 200.

As shown in FIG. 13, device 400 is preferably surgically placed directly on the outer surface of sclera 100 below Tenon's capsule 101 with well 216 and inner core 220 directly over the area of sclera 100 above macula 98. Most preferably, inner core 220 is directly over the area of sclera 100 above fovea 117. Device 400 has a generally trapezoidal geometry when viewed from its orbital surface 204. Device 400 also has a first extension 404 and a second extension 406 designed to accommodate the superior border 408 and the inferior border 410 of insertion 111 of lateral rectus muscle 105. Due to the geometry of device 400, anchoring extensions 404 and 406 to insertion 111 of lateral rectus muscle 105 in this manner automatically locates inner core 220 over macula 98 and fovea 117. Anchoring extensions 404 and 406 to insertion 111 of lateral rectus muscle 105 in this manner also helps to immobilize and prevent migration of device 400 after implantation.

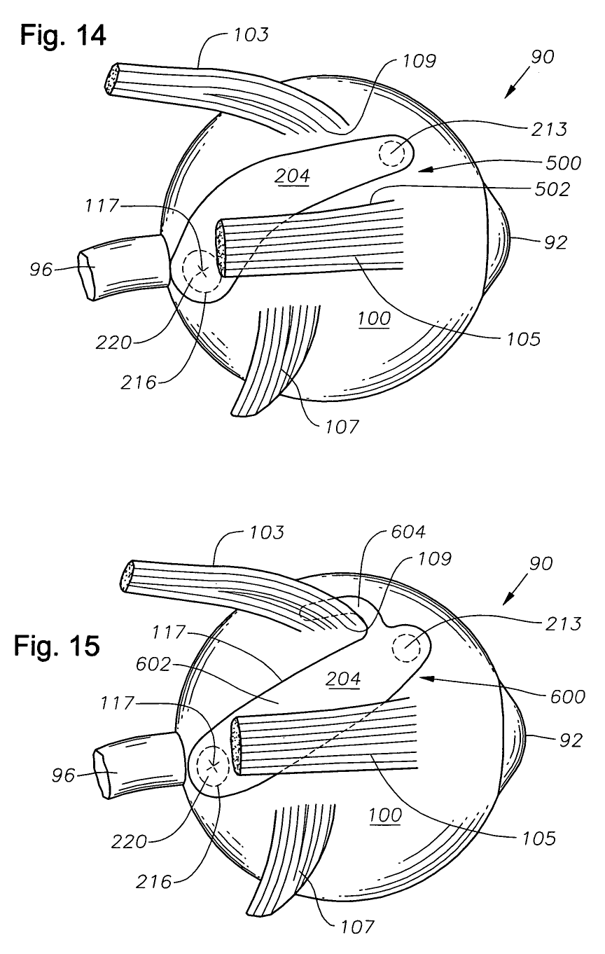

As shown in FIG. 14, device 500 is preferably surgically placed directly on the outer surface of sclera 100 below Tenon's capsule 101 with well 216 and inner core 220 directly over the area of sclera 100 above macula 98. Most preferably, inner core 220 is directly over the area of sclera 100 above fovea 117. Device 500 has a generally club-shaped or arc-shaped geometry when viewed from its orbital surface 204, which is designed to facilitate implantation between insertion 109 of superior rectus muscle 103 and superior border 502 of lateral rectus muscle 105.

As shown in FIG. 15, device 600 is preferably surgically placed directly on the outer surface of sclera 100 below Tenon's capsule 101 with well 216 and inner core 220 directly over the area of sclera 100 above macula 98. Most preferably, inner core 220 is directly over the area of sclera 100 above fovea 117. Device 600 has a generally elliptical or rectangular geometry when viewed from its orbital surface 204. Device 600 also has an extension 604 extending from body 602. Extension 604 is disposed on the outer surface of sclera 100 and beneath superior rectus muscle 103 proximate to, or contacting, insertion 109 of superior rectus muscle 103. Due to the geometry of device 600, anchoring extension 604 to insertion 109 of superior rectus muscle 103 in this manner automatically locates inner core 220 over macula 98 and fovea 117. Anchoring extension 604 to insertion 109 of superior rectus muscle 103 in this manner also helps to immobilize and prevent migration of device 600 after implantation.

From the above, it may be appreciated that the present invention provides improved devices and methods for safe, effective, rate-controlled, localized delivery of a variety of pharmaceutically active agents to the eye, and particularly to the posterior segment of the eye to combat ARMD, CNV, retinopathies, retinitis, uveitis, macular edema, glaucoma, and neuropathies. The surgical procedure for implanting such devices is safe, simple, quick, and capable of being performed in an outpatient setting. Such devices are easy and economical to manufacture. Furthermore, because of their capability to deliver a wide variety of pharmaceutically active agents, such devices are useful in clinical studies to deliver various ophthalmic agents that create a specific physical condition in a patient.

It is believed that the operation and construction of the present invention will be apparent from the foregoing description. While the apparatus and methods shown or described above have been characterized as being preferred, various changes and modifications may be made therein without departing from the spirit and scope of the invention as defined in the following claims.

What is claimed is:

1. A drug delivery device for an eye, said eye comprising a sclera, a macula, and an extraocular muscle, comprising:
a pharmaceutically active agent; and
a body having an extension for accomodating said extraocular muscle;
whereby when said device is disposed on an outer surface of said sclera so that said extension accomodates said extraocular muscle, said pharmaceutically active agent is disposed proximate said macula.

2. The drug delivery device of claim 1 whereby when said device is disposed on an outer surface of said sclera so that said extension accomodates said extraocular muscle, said pharmaceutically active agent is disposed above said macula.

3. The drug delivery device of claim 1 wherein said extraocular muscle is an inferior oblique muscle.

4. The drug delivery device of claim 1 wherein said extraocular muscle is a lateral rectus muscle.

5. The drug delivery device of claim 1 wherein said extraocular muscle is a superior rectus muscle.

6. A drug delivery device for an eye, said eye comprising a sclera and an extraocular muscle, comprising:
a pharmaceutically active agent; and
a body having an extension for accomodating said extraocular muscle;
whereby when said device is disposed on an outer surface of said sclera so that said extension accomodates said extraocular muscle, said extension helps to immobilize and to prevent migration of said device.

7. The drug delivery device of claim 6 wherein said extraocular muscle is an inferior oblique muscle.

8. The drug delivery device of claim 6 wherein said extraocular muscle is a lateral rectus muscle.

9. The drug delivery device of claim 6 wherein said extraocular muscle is a superior rectus muscle.

10. A drug delivery device for an eye, said eye comprising an extraocular muscle, comprising:
a pharmaceutically active agent; and
a body having an extension for accomodating said extraocular muscle, said extension being capable of extending from said body in a first position so as to accommodate said extraocular muscle, and said extension being capable of folding above or beneath said body in a second position so as to facilitate implantation of said device.

11. A drug delivery device for an eye, said eye comprising a sclera, comprising:
a pharmaceutically active agent; and
a body having a scleral surface for contacting said sclera and an immobilizing structure disposed on said scleral surface.

12. The drug delivery device of claim 11 wherein said immobilizing structure is a suction cup.

13. The drug delivery device of claim 11 wherein said immobilizing structure is a bioadhesive coating.

14. The drug delivery device of claim 11 wherein said immobilizing structure is a region containing a sharp prong.

15. A drug delivery device for an eye, said eye comprising a sclera, a macula, a superior rectus muscle, and a lateral rectus muscle, comprising:
a body having:
a scleral surface;
a well having an opening to said scleral surface; and
a geometry that facilitates an implantation of said device on an outer surface of said sclera, between said superior rectus muscle and said lateral rectus muscle, beneath said lateral rectus muscle, and with said well disposed proximate said macula; and
an inner core disposed in said well and comprising a pharmaceutically active agent.

16. The drug delivery device of claim 15 wherein said inner core is a tablet.

17. The drug delivery device of claim 15 wherein said eye comprises a Tenon's capsule, and said geometry facilitates an implantation of said device beneath said Tenon's capsule.

18. The drug delivery device of claim 15 wherein said geometry facilitates an implantation of said device with said well disposed above said macula.

19. The drug delivery device of claim 18 wherein said macula comprises a fovea, and said geometry facilitates an implantation of said device with said well disposed above said fovea.

20. The drug delivery device of claim 15 wherein said body comprises an orbital surface, and said geometry is a generally club-shaped geometry when viewed from said scleral surface or said orbital surface.

21. The drug delivery device of claim 15 wherein said body comprises an orbital surface, and said geometry is a generally arc-shaped geometry when viewed from said scleral surface or said orbital surface.

22. The drug delivery device of claim 15 wherein said scleral surface has a radius of curvature that facilitates contact with said sclera.

23. A method of delivering a pharmaceutically active agent to an eye, said eye comprising a sclera, a macula, a superior rectus muscle, and a lateral rectus muscle, comprising the steps of:
providing a drug delivery device comprising:
a pharmaceutically active agent; and
a body having a geometry that facilitates an implantation of said device on an outer surface of said sclera, between said superior rectus muscle and said lateral rectus muscle, beneath said lateral rectus muscle, and with said pharmaceutically active agent disposed proximate said macula; and
disposing said device on an outer surface of said sclera, between said superior rectus muscle and said lateral rectus muscle, beneath said lateral rectus muscle, and with said pharmaceutically active agent disposed proximate said macula.

24. The method of claim 23 wherein said eye comprises a Tenon's capsule, and said disposing step comprises disposing said device below said Tenon's capsule.

25. The method of claim 23 wherein said disposing step comprises disposing said pharmaceutically active agent above said macula.

26. The method of claim 25 wherein said disposing step comprises disposing said pharmaceutically active agent above said fovea.

27. The method of claim 23 wherein said body comprises a scleral surface and an orbital surface, and said geometry is a generally club-shaped geometry when viewed from said scleral surface or said orbital surface.

28. The method of claim 23 wherein said body comprises a scleral surface and an orbital surface, and said geometry is a generally arc-shaped geometry when viewed from said scleral surface or said orbital surface.

29. The method of claim 23 wherein said device comprises an inner core comprising said pharmaceutically active agent, and said body comprises a scleral surface and a well having an opening to said scleral surface for receiving said inner core.

30. The method of claim 29 wherein said inner core is a tablet.
